(19) United States
(12) Patent Application Publication
(10) Pub. No.: US 2016/0118589 A1
(43) Pub. Date: Apr. 28, 2016

(54) Organic Semiconductor Film, Method for Manufacturing Same, and Transistor Structure

(71) Applicant: TOKYO ELECTRON LIMITED, Tokyo (JP)

(72) Inventors: Munehito KAGAYA, Tokyo (JP); Takashi MATSUMOTO, Nirasaki City, Yamanashi (JP); Taiki KATO, Nirasaki City, Yamanashi (JP)

(21) Appl. No.: 14/895,404

(22) PCT Filed: May 27, 2014
(86) PCT No.: PCT/JP2014/064563

§ 371 Date: Dec. 2, 2015

(30) Foreign Application Priority Data
Jun. 3, 2013 (JP) 2013-116758

(51) Int. Cl. H01L 51/00; C07C 13/70; C07C 13/64; C07C 2/76

(52) U.S. Cl. CPC H01L 51/0056; H01L 51/0057; C07C 2/76; C07C 13/70; C07C 13/64; H01L 51/0558

(57) ABSTRACT

Provided is an organic semiconductor film with which a desired band gap can be securely achieved. In an ultrahigh vacuum film formation device (10), 5,5′,5″,5′″,5″″,5′″″-hexabromocyclohexa-m-phenylene (CHP) powder is made to sublimate from a fuel cell (12) by the application of heat energy, bromine is made to separate out by causing the CHP molecules to collide with a catalyst metal layer (M) of a substrate (G), and a plurality of generated phenyl radicals are made to mutually bond through Ullmann reactions, thereby forming a two-dimensional network structure of carbon atoms.

### TECHNICAL FIELD

The present disclosure relates to an organic semiconductor film, a method for manufacturing the same, and a transistor structure, in particular, to an organic semiconductor film having a pseudo graphene structure.

### BACKGROUND

While metals, for example, Cu, have been used for the wiring of transistor structures, ultrafine wiring structures formed by metal wiring materials such as Cu have the problem that the wire has high resistivity because conducting electrons are strongly affected by the inelastic scattering at interfaces due to the fine line effect.

Meanwhile, graphene has an extremely long mean free path and a high mobility, and there has been suggested the possibility of realizing low-resistant wirings outperforming Cu when graphene is applied to fine wiring structures (e.g., see Non-Patent Document 1). Accordingly, using graphene instead of Cu in wiring films is considered for the next generations of transistor structure which need to achieve finer layered structures or wiring structures.

In the CVD method (for example, thermal CVD or plasma CVD), which is a typical process for producing graphene, the surface of a substrate is covered with a catalyst metal layer and the catalyst metal layer is activated, followed by dissolution of carbon atoms dissociated from the source gas into the catalyst metal layer, which was activated once, and recrystallization of the carbon atoms. That is, CVD can be easily adapted for conventional semiconductor device forming processes because it allows the direct production of graphene on a substrate with a relatively large area.

Among the CVD methods, thermal CVD requires heating a substrate up to about 1000 degrees C. in order to thermally dissociate the source gas, which may cause the degradation of other wiring films or insulating films in the transistor structure. As a result, under the current circumstances, plasma CVD is most commonly used because it requires the substrate to be heated up to a relatively low temperature, e.g., just 600 degrees C. or lower, by dissociating the source gas by plasma. In the plasma CVD method, hydrocarbon-based gases, for example, are used as the source gas, and plasma is generated from the hydrocarbon-based gas, after which carbon radicals in the plasma are dissolved into a catalyst metal layer (e.g., see Patent Document 1).

Meanwhile, graphene cannot perform the switching operation when used in channels because graphene is a semi-metal and cannot turn off the current flowing through itself However, various methods have been proposed to overcome this difficulty and generate a band gap in graphene that is required for performing the switching operation.

Specifically, methods have been proposed where narrow regions are formed by processing thin-film graphene generated by the plasma CVD described above into nanoscale rectangular shape to form graphene nanoribbons, or by creating holes according to a specific pattern in the graphene to form a graphene nanomesh, thereby generating a band gap according to the quantum size effect (e.g., see Non-Patent Documents 2 to 4). For example, it has been reported that in order to generate a band gap of 500 meV by the quantum size effect, graphene nanoribbons need to be processed so as to have a width not greater than 8 nm (e.g., see Non-Patent Document 5).

### PRIOR ART DOCUMENTS

### Patent Documents

Patent Document 1: Japanese Laid-Open Publication No. 2010-212619

### Non-Patent Documents

Non-Patent Document 1: A. Naeemi and J. D. Meindl, IEEE EDL, 28, p. 428 (2007)

Non-Patent Document 2: X. Wang et al., Phys. Rev. Lett., 100, 206803 (2008)

Non-Patent Document 3: J. Bai et al., Nat. Nanotechnol., 5, 190 (2010)

Non-Patent Document 4: W. Oswald et al., Phys. Rev. B, 85, 115431 (2012)

Non-Patent Document 5: F. Schwierz et al., Nat. Nanotechnol., 5, 486 (2010)

### SUMMARY

Thus, in order to obtain a desired band gap, lithography on the order of Å (Angstrom) should be performed on graphene, which is difficult to achieve.

The present disclosure provides an organic semiconductor film with which a desired band gap can be reliably obtained, a method for manufacturing the same, and a transistor structure.

In order to solve the above problems, the present disclosure provides an organic semiconductor film having a pseudo graphene structure formed by contiguous extension of a two-dimensional network structure represented by formula (I).

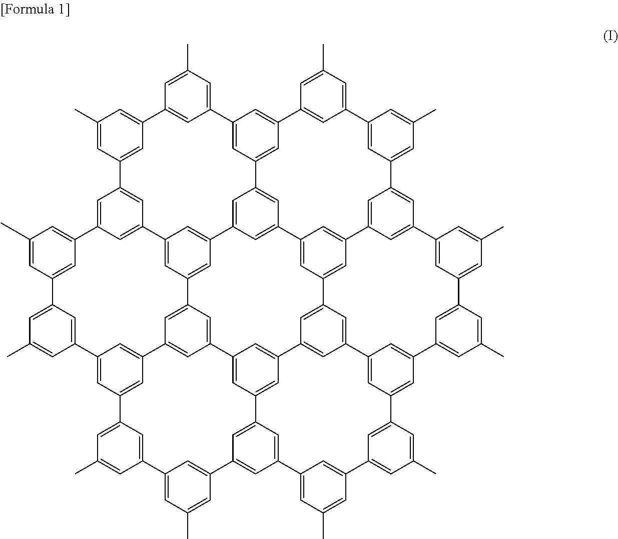

In the present disclosure, the pseudo graphene structure is preferably comprised of a monolayer of the two-dimensional network structure.

In order to solve the above problems, the present disclosure provides an organic semiconductor film having a pseudo graphene structure formed by contiguous extension of a two-dimensional network structure represented by formula (II).

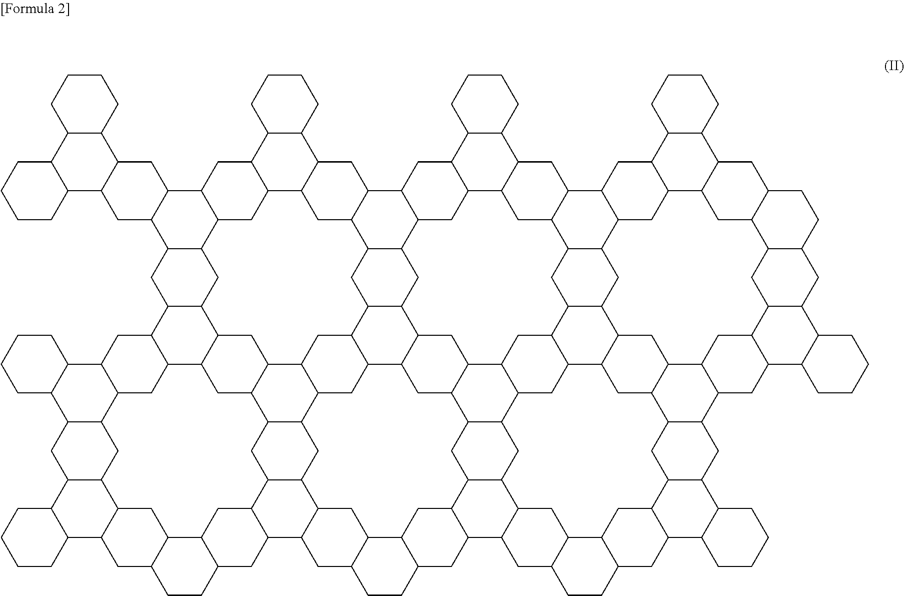

In the present disclosure, the pseudo graphene structure is preferably comprised of a monolayer of the two-dimensional network structure.

In order to solve the above problems, the present disclosure provides a method for preparing an organic semiconductor film, wherein the two-dimensional network structure in the aforementioned is formed by polymerizing a plurality of 5,5′,5″,5′″,5″″,5′″″-hexabromocyclohexa-m-phenylene (hereinafter, “CHP”) and the CHP has bromine at its side chains.

In the present disclosure, the two-dimensional network structure is preferably formed by depositing the plurality of CHPs on the surface of a single-crystalline metal having catalytic activity.

In the present disclosure, when the single-crystalline metal has a face centered cubic lattice, the surface of the single-crystalline metal is preferably composed of the (111) plane of the face centered cubic lattice.

In the present disclosure, when the single-crystalline metal has a hexagonal close packed structure, the surface of the single-crystalline metal is preferably composed of the (0001) plane of the hexagonal close packed structure.

In the present disclosure, the two-dimensional network structure is preferably formed by depositing the plurality of CHPs on the surface of a polycrystalline metal containing grains having catalytic activity.

In the present disclosure, when the polycrystalline metal has a face centered cubic lattice, the surface of the grains is preferably composed of the (111) plane of the face centered cubic lattice.

In the present disclosure, when the polycrystalline metal has a hexagonal close packed structure, the surface of the grains is preferably composed of the (0001) plane of the hexagonal close packed structure.

In order to solve the above problems, the present disclosure provides a method for manufacturing an organic semiconductor film, wherein the two-dimensional network structure in the aforementioned organic semiconductor film-is formed by polymerizing a plurality of 2,3,6,7,10,11-hexabromotriphenylene (hereinafter, “HBTP”).

In the present disclosure, the two-dimensional network structure is preferably formed by depositing the plurality of HBTPs on the surface of a single-crystalline metal having catalytic activity.

In the present disclosure, when the single-crystalline metal has a face centered cubic lattice, the surface of the single-crystalline metal is preferably composed of the (111) plane of the face centered cubic lattice.

In the present disclosure, when the single-crystalline metal has a hexagonal close packed structure, the surface of the single-crystalline metal is preferably composed of the (0001) plane of the hexagonal close packed structure.

In the present disclosure, the two-dimensional network structure is preferably formed by depositing the plurality of HBTPs on the surface of polycrystalline metal comprised of grains having catalytic activity.

In the present disclosure, when the polycrystalline metal has a face centered cubic lattice, the surface of the grains is preferably composed of the (111) plane of the face centered cubic lattice.

In the present disclosure, when the polycrystalline metal has a hexagonal close packed structure, the surface of the grains is preferably composed of the (0001) plane of the hexagonal close packed structure.

In order to solve the above problems, the present disclosure provides a transistor structure wherein the organic semiconductor film-is used for channels.

According to the present disclosure, an organic semiconductor film has a pseudo graphene structure which is formed by contiguous extension of a two-dimensional network structure of molecules of an organic compound. However, since this two-dimensional network structure is formed based on the spontaneous order of the molecules of the organic compound, the neck or the shape of steps in the two-dimensional network structure is uniquely determined according to the shape or side groups of the organic compound molecules. Since these necks are composed according to the valence of each atom, the width of the neck can be set to be in the order of Å. In other words, since necks with a width in the order of Å can be uniquely formed based on the spontaneous order of the molecules of the respective organic compounds, a desired band gap can be securely achieved.

### BRIEF DESCRIPTION OF THE DRAWINGS

FIG. 1 is a sectional view schematically illustrating a film forming device for manufacturing an organic semiconductor film according to the first embodiment of the present disclosure.

FIG. 2 is a graph showing the band structure of a pseudo graphene structure having a two-dimensional network structure formed by the polymerization of CHPs.

FIG. 3 is a graph showing the band structure of a pseudo graphene structure having a two-dimensional network structure formed by the polymerization of HBTPs.

FIG. 4 is a sectional view schematically illustrating the configuration of an example of a bottom-gated thin-film transistor structure where an organic semiconductor film according to the first and second embodiments of the present disclosure is applied.

FIG. 5 is a sectional view schematically illustrating the configuration of the first variant example of a bottom-gated thin-film transistor structure where an organic semiconductor film according to each embodiment of the present disclosure is applied.

FIG. 6 is a sectional view schematically illustrating the configuration of the second variant example of a bottom-gated thin-film transistor structure where an organic semiconductor film according to each embodiment of the present disclosure is applied.

FIG. 7 is a sectional view schematically illustrating the configuration of the third variant example of a bottom-gated thin-film transistor structure where an organic semiconductor film according to each embodiment of the present disclosure is applied.

FIG. 8 is a sectional view schematically illustrating the configuration of an example of a top-gated thin-film transistor structure where an organic semiconductor film according to each embodiment of the present disclosure is applied.

FIG. 9 is a sectional view schematically illustrating the configuration of the first variant example of a top-gated thin-film transistor structure where an organic semiconductor film according to each embodiment of the present disclosure is applied.

FIG. 10 is a sectional view schematically illustrating the configuration of the second variant example of a top-gated thin-film transistor structure where an organic semiconductor film according to each embodiment of the present disclosure is applied.

### DETAILED DESCRIPTION

Hereinafter, the embodiments of the present disclosure will be described in detail with reference to the accompanying drawings.

First, explanations will be provided regarding an organic semiconductor film according to the first embodiment of the present disclosure.

The organic semiconductor film according to this embodiment is manufactured from CHP, a molecule of the organic compound represented by formula (III).

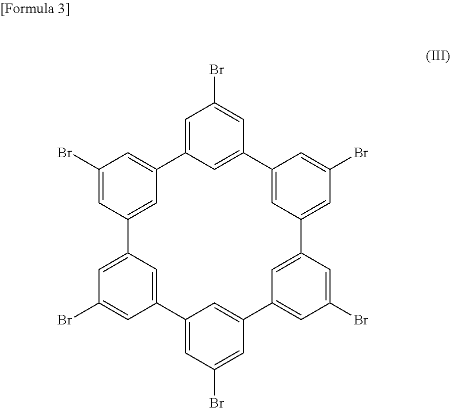 [SMILES: Brc1cc2cc(c1)c1cc(Br)cc(c1)c1cc(Br)cc(c1)c1cc(Br)cc(c1)c1cc(Br)cc(c1)c1cc(Br)cc2c1]

The CHP represented by formula (III) above has bromine, as a halogen, at its side chains. In the present embodiment, a two-dimensional network structure represented by formula (I) below is formed from a plurality of CHPs represented by formula (III) above. Hereinafter, the CHP in the present embodiment has bromine at its side chains, unless stated otherwise.

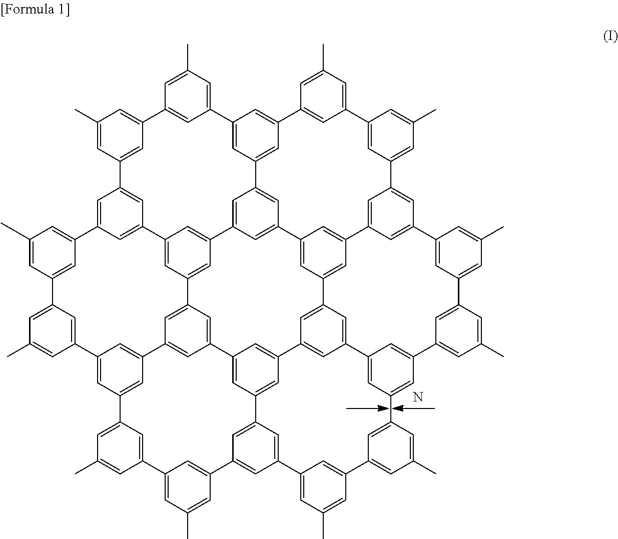

In an organic semiconductor film according to the present embodiment, a pseudo graphene structure is formed by contiguously extending the two-dimensional network structure represented by formula (I) (hereinafter, “first two-dimensional network structure”). In the first two-dimensional network structure, a neck is a region of atomic bonding which links two adjacent phenyl groups (indicated by “N” in formula (I) above), and the width of the neck is in the order of Å.

Since a two-dimensional network structure formed from molecules of an organic compound is usually formed based on the spontaneous order of the molecules of the organic compound, the neck width or the shape of steps in the two-dimensional network structure is determined according to the shape or side groups of the organic compound molecules. Accordingly, the first two-dimensional network structure is also uniquely determined according to the shape or side groups of CHP, as does the neck of the structure. That is, when an organic semiconductor film is manufactured by forming the first two-dimensional network structure from a plurality of CHPs and obtaining a pseudo graphene structure, necks with widths in the order of Å can be definitely formed, and as a result, a desired band gap can be securely achieved by the quantum size effect in the necks.

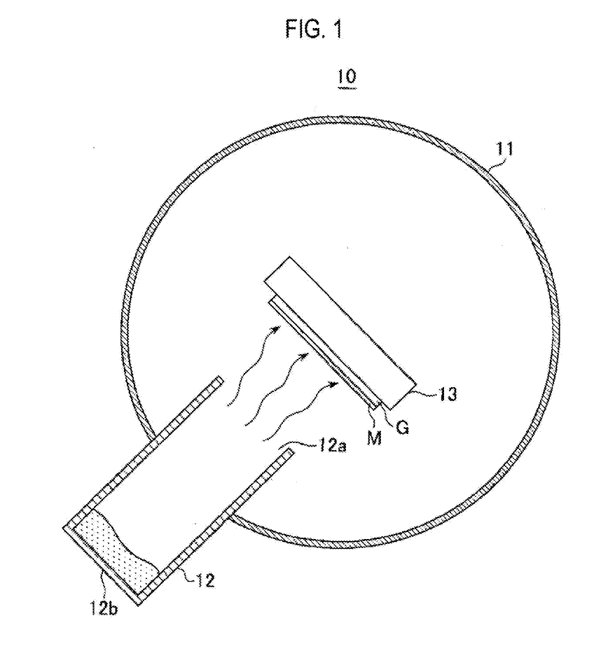

FIG. 1 is a sectional view schematically illustrating the configuration of a film forming device for manufacturing an organic semiconductor film according to the present embodiment.

Referring to FIG. 1, ultrahigh vacuum film forming device 10 includes chamber 11 and source material cell 12 installed in chamber 11. In chamber 11, stage 13, on which substrate G comprising of, for example, a silicon substrate, glass substrate, or plastic (polymer) substrate can be mounted, is located. Stage 13 has a heater (not shown) enclosed therein to heat mounted substrate G. Also, chamber 11 has a pumping element (not shown) which exhausts air in chamber 11 to reduce the pressure.

Source material cell 12, having a cylindrical body with an open end, is installed in chamber 11 such that open end 12a faces stage 13, and powder or the like of an organic compound (for example, CHP) is placed inside the cell. Source material cell 12 also has a heater (not shown) near closed end 12b, and the heater heats the organic compound contained in the cell body to make it sublimate.

In ultrahigh vacuum film forming device 10, the sublimated organic compound enters chamber 11 and then gets deposited on the surface of catalyst metal layer M formed on the surface of substrate G. Catalyst metal layer M consists of a single-crystalline metal having catalytic activity, for example, a transition metal having a face centered cubic lattice (Cu, Ni, Au, etc.) or a transition metal having a hexagonal close packed structure (Co, Ru, etc.), or an alloy comprising such a transition metal. When the single-crystalline metal is a transition metal having a face centered cubic lattice, the surface of catalyst metal layer M is composed of the (111) plane which is the most closely packed plane of the face centered cubic lattice, and when the single-crystalline metal is a transition metal having a hexagonal close packed structure, the surface of catalyst metal layer M is composed of the (0001) plane which is the most closely packed plane of the hexagonal close packed structure.

A plurality of CHP molecules loaded with heat energy from source material cell 12 collide with catalyst metal layer M, and bromine is dissociated from the CHP molecules due to the energy of each molecule and the catalysis by catalyst metal layer M, generating a plurality of phenyl radicals. Each phenyl radical is polymerized by the Ullmann reaction resulting from the catalysis of catalyst metal layer M, heating from the heater in stage 13 and the energy in each molecule. Here, the phenyl radicals are polymerized based on the spontaneous order of the CHP molecules, thereby forming the first two-dimensional network structure. In the present embodiment, the collision of CHP molecules with catalyst metal layer M or the elimination of bromine from the CHP molecules occurs in the gas phase.

When each phenyl radical is polymerized, since the elimination of bromine and Ullman reaction are interfacial reactions, only those CHP molecules that are in direct contact with the surface of catalyst metal layer M undergo the reactions. Furthermore, in the Ullman reaction, due to the easy lattice match between the phenyl honeycomb structure of each phenyl radical and the most closely packed plane constituting the surface of catalyst metal layer M (for example, the (111) plane when catalyst metal layer M is formed from a transition metal having a face centered cubic lattice, and the (0001) plane when the catalyst metal layer M is formed from a transition metal having a hexagonal close packed structure), the polymerization of phenyl radicals progresses along the surface of catalyst metal layer M. Consequently, the first two-dimensional network structure formed on the surface of catalyst metal layer M takes the form of a monolayer.

In the present embodiment, catalyst metal layer M may consist of, rather than a single-crystalline metal, a polycrystalline metal comprised of grains, having catalytic activity, for example, a transition metal having a face centered cubic lattice (Cu, Ni, Au, etc.) or a transition metal having a hexagonal close packed structure (Co, Ru, etc.), or an alloy comprising such a transition metal, wherein when the polycrystalline metal is a transition metal having a face centered cubic lattice, the surface of the grains is composed of the (111) plane which is the most closely packed plane of the face centered cubic lattice and when the polycrystalline metal is a transition metal having a hexagonal close packed structure, the surface of the grains is composed of the (0001) plane which is the most closely packed plane of the hexagonal close packed structure. In this case as well, catalysis brings about the dissociation of bromine and the phenyl radicals are polymerized. However, due to the easy lattice match between the phenyl honeycomb structure of each phenyl radical and the most closely packed plane constituting the surface of the grains, the polymerization of phenyl radicals progresses along the surface of the grains, producing the first two-dimensional network structure in the form of a monolayer.

To form the first two-dimensional network structure, using a CHP having iodine, as a halogen, at its side chains rather than a CHP having bromine at its side chains can also be contemplated. However, it is not desirable to use a CHP having iodine for obtaining a pseudo graphene structure consisting of the first two-dimensional network structure, because there are concerns that iodine dissociated during the formation of the first two-dimensional network structure might combine with each other to remain as a solid on the surface of catalyst metal layer M as iodine has a lower vapor pressure than bromine.

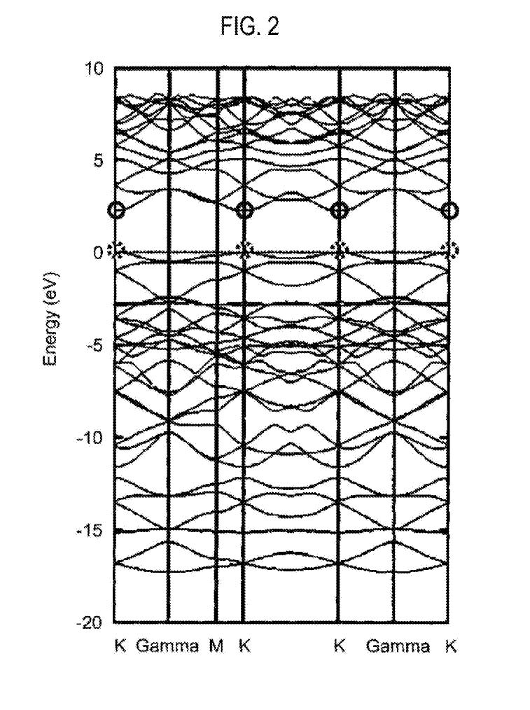

Next, the present inventors calculated the band gap of the pseudo graphene structure consisting of the first two-dimensional network structure using first principles calculations based on density functional theory. As shown in FIG. 2, the bottom of the conduction band (represented by solid line circles in the graph) and the top of the valence band (represented by dotted line circles in the graph) were separated in the band structure, demonstrating the opening of a band gap. In the band structure shown in FIG. 2, the band gap was 2.27 eV. Thus, it can be seen that an organic semiconductor film having a pseudo graphene structure consisting of the first two-dimensional network structure exhibits semiconductor properties necessary for realizing the switching operation.

The present inventors also calculated, using quantum chemistry, the energy required for the dissociation of bromine from organic compounds. In particular, the energy required for dissociating bromine (Br₂) from the organic compounds bromobenzene (C₆H₅Br) or tetra(4-bromophenyl)porphyrin (Br₄TPP) and the dissociation energy for bromine radicals were calculated to be about 3 eV or greater. Thus, it can be seen that the dissociation of bromine or bromine radicals from an organic compound requires high energy, and since heating of the organic compound alone can hardly bring about the dissociation of bromine, catalysis by a catalyst metal or more energy input (for example, light energy by laser irradiation) is necessary.

Next, an explanation will be provided regarding an organic semiconductor film according to the second embodiment of the present disclosure.

The organic semiconductor film according to this embodiment is manufactured from HBTP, a molecule of an organic compound represented by formula (IV).

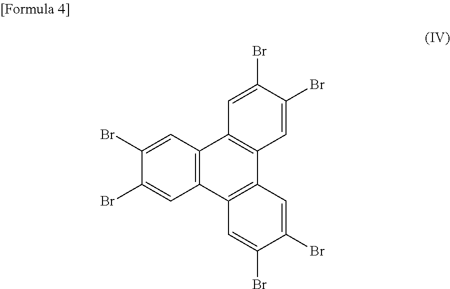 [SMILES: Brc1cc2c3cc(Br)c(Br)cc3c3cc(Br)c(Br)cc3c2cc1Br]

The HBTP represented by formula (IV) has bromine, as a halogen, at its side chains. In the present embodiment, a two-dimensional network structure represented by formula (II) below is formed from a plurality of HBTPs represented by formula (IV) above.

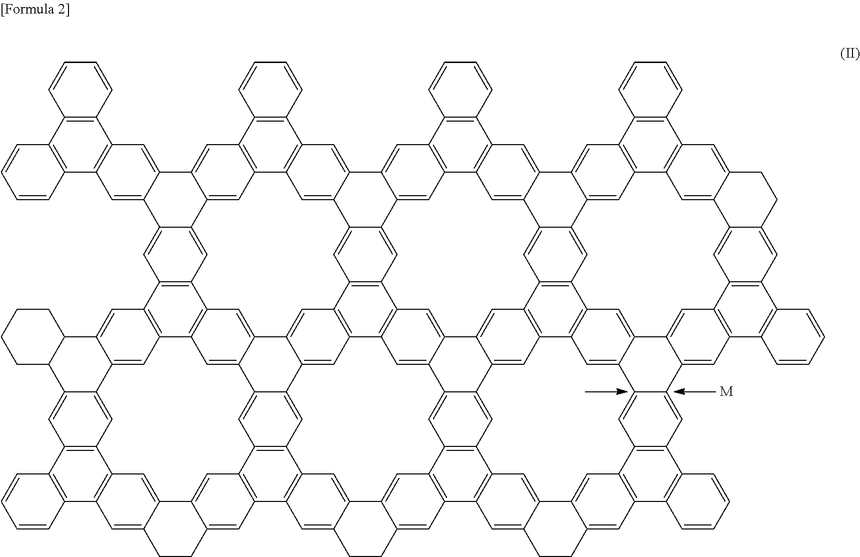

In an organic semiconductor film according to the present embodiment, a pseudo graphene structure is formed by contiguously extending the two-dimensional network structure represented by formula (II) (hereinafter, “second two-dimensional network structure”). In the second two-dimensional network structure, a neck is the region between two adjacent phenyl groups (indicated by “M” in formula (II)), and the width of the neck is in the order of Å, being about 1.42 Å which is equal to the bond length between the carbon atoms in a phenyl group.

As in the first two-dimensional network structure, the neck in the second two-dimensional network structure is also uniquely determined according to the shape or side groups of HBTP. Thus, when an organic semiconductor film is manufactured by forming the second two-dimensional network structure from a plurality of HBTPs to obtain a pseudo graphene structure, necks with widths in the order of Å can be definitely formed, and as a result, a desired band gap can be securely achieved by the quantum size effect in the necks.

Ultrahigh vacuum film forming device 10 in FIG. 1 is also used for manufacturing an organic semiconductor film from HBTP. As in the first embodiment, the phenyl radicals generated by the dissociation of bromine due to catalysis by catalyst metal layer M and the like are polymerized by the Ullmann reaction. When each phenyl radical is polymerized, since the elimination of bromine and Ullman reaction are interfacial reactions, only those HBTP molecules that are in direct contact with catalyst metal layer M undergo the reactions. Furthermore, in the Ullman reaction, due to the easy lattice match between the phenyl honeycomb structure of each phenyl radical and the most closely packed plane constituting the surface of catalyst metal layer M (for example, the (111) plane when catalyst metal layer M is formed from a transition metal having a face centered cubic lattice, and the (0001) plane when catalyst metal layer M is formed from a transition metal having a hexagonal close packed structure), the polymerization of phenyl radicals progresses along the surface of catalyst metal layer M. Consequently, the second two-dimensional network structure formed on the surface of catalyst metal layer M takes the form of a monolayer.

In the present embodiment also, as in the first embodiment, catalyst metal layer M may consist of, rather than a single-crystalline metal, a polycrystalline metal comprised of grains and having catalytic activity, for example, a transition metal having a face centered cubic lattice (Cu, Ni, Au, etc.) or a transition metal having a hexagonal close packed structure (Co, Ru, etc.), or an alloy comprising such a transition metal, wherein when the polycrystalline metal is a transition metal having a face centered cubic lattice, the surface of the grains is composed of the (111) plane which is the most closely packed plane of the face centered cubic lattice and when the polycrystalline metal is a transition metal having a hexagonal close packed structure, the surface of the grains is composed of the (0001) plane which is the most closely packed plane of the hexagonal close packed structure. In this case as well, the polymerization of phenyl radicals progresses along the surface of the grains, producing the second two-dimensional network structure in the form of a monolayer.

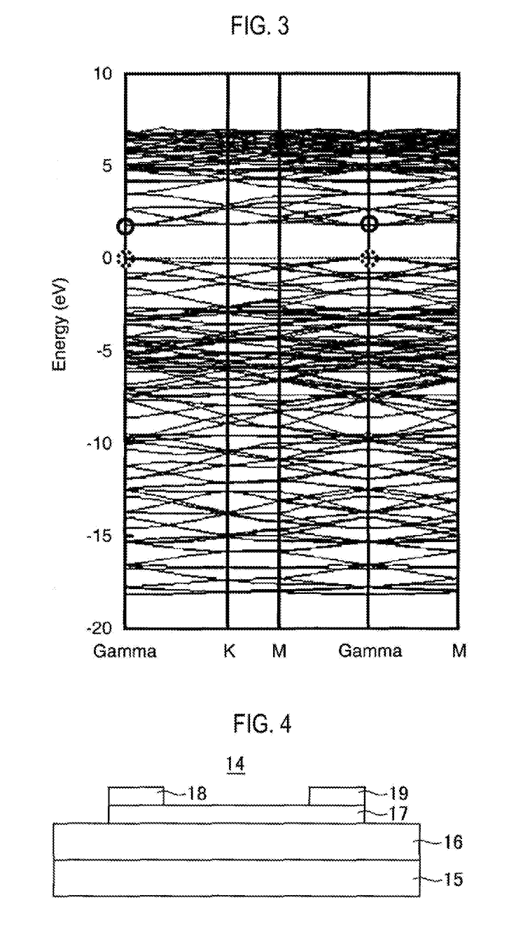

Next, the present inventors, as in the first embodiment, caluclated the band gap of the pseudo graphene structure consisting of the second two-dimensional network structure using first principles calculations based on density functional theory. As shown in FIG. 3, the bottom of the conduction band (represented by solid line circles in the graph) and the top of the valence band (represented by dotted line circles in the graph) were separated in the band structure, demonstrating the opening of a band gap. In the band structure shown in FIG. 3, the band gap was 1.77 eV. Thus, it can be seen that an organic semiconductor film having a pseudo graphene structure consisting of the second two-dimensional network structure also exhibits semiconductor properties necessary for realizing the switching operation.

Hereinafter, an explanation will be provided regarding the configurations of the thin-film transistor structure to which an organic semiconductor film according to each embodiment is applied.

First, configurations of a bottom-gated thin-film transistor structure will be described.

FIG. 4 is a sectional view schematically illustrating the configuration of an example of a bottom-gated thin-film transistor structure where an organic semiconductor film according to an embodiment of the present disclosure is applied.

Referring to FIG. 4, thin-film transistor structure 14 comprises gate electrode 15 formed on a substrate (not shown); gate-insulating film 16 formed on gate electrode 15; channel layer 17 formed on gate-insulating film 16; and source electrode 18 and drain electrode 19 formed on channel layer 17. Channel layer 17 is comprised of an organic semiconductor film according to the first or second embodiment.

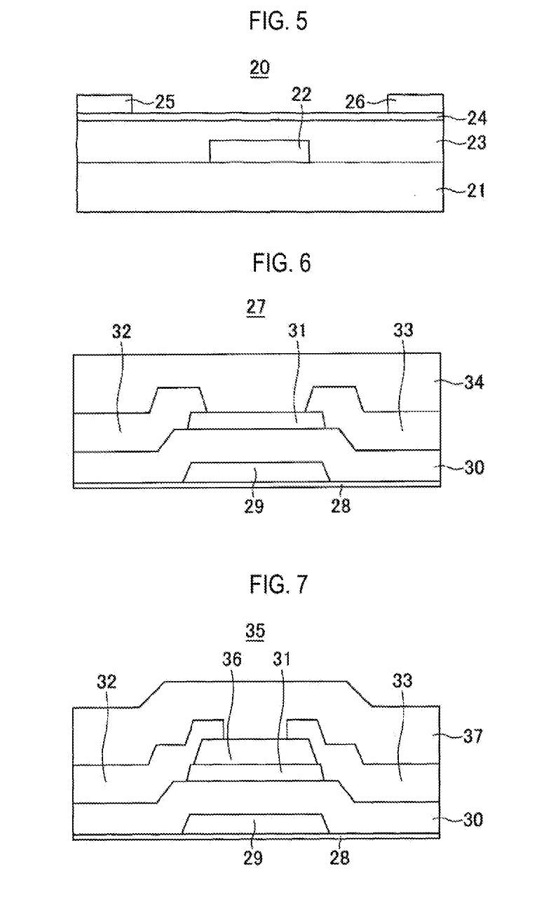

FIG. 5 is a sectional view schematically illustrating the configuration of the first variant example of a bottom-gated thin-film transistor structure where an organic semiconductor film according to each embodiment of the present disclosure is applied.

Referring to FIG. 5, thin-film transistor structure 20 comprises gate electrode 22 formed on substrate 21; gate-insulating film 23 formed so as to cover substrate 21 and gate electrode 22; channel layer 24 formed on gate-insulating film 23; and source electrode 25 and drain electrode 26 formed on channel layer 24. Channel layer 24 is comprised of an organic semiconductor film according to the first or second embodiment.

FIG. 6 is a sectional view schematically illustrating the configuration of the second variant example of a bottom-gated thin-film transistor structure where an organic semiconductor film according to each embodiment of the present disclosure is applied.

Referring to FIG. 6, thin-film transistor structure 27 comprises undercoat layer 28 formed as a film on a substrate (not shown); gate electrode 29 formed on a part of undercoat layer 28; gate-insulating film 30 formed so as to cover undercoat layer 28 and gate electrode 29; channel layer 31 formed on gate-insulating film 30 to be located directly above gate electrode 29; source electrode 32 and drain electrode 33 respectively formed on gate-insulating film 30 on opposite sides of channel layer 31; and passivation layer 34 formed so as to cover channel layer 31, source electrode 32 and drain electrode 33. Channel layer 31 is comprised of an organic semiconductor film according to the first or second embodiment.

FIG. 7 is a sectional view schematically illustrating the configuration of the third variant example of a bottom-gated thin-film transistor structure where an organic semiconductor film according to each embodiment of the present disclosure is applied.

Referring to FIG. 7, thin-film transistor structure 35 comprises undercoat layer 28 formed as a film on a substrate (not shown); gate electrode 29 formed on a part of undercoat layer 28; gate-insulating film 30 formed so as to cover undercoat layer 28 and gate electrode 29; channel layer 31 formed on gate-insulating film 30 to be located directly above gate electrode 29; source electrode 32 and drain electrode 33 respectively formed on gate-insulating film 30 on opposite sides of channel layer 31; etching stopper layer 36 formed so as to cover channel layer 31; and passivation layer 37 formed so as to cover etching stopper layer 36, source electrode 32 and drain electrode 33. Channel layer 31 is comprised of an organic semiconductor film according to the first or second embodiment.

Next, configurations of a top-gated thin-film transistor structure will be described.

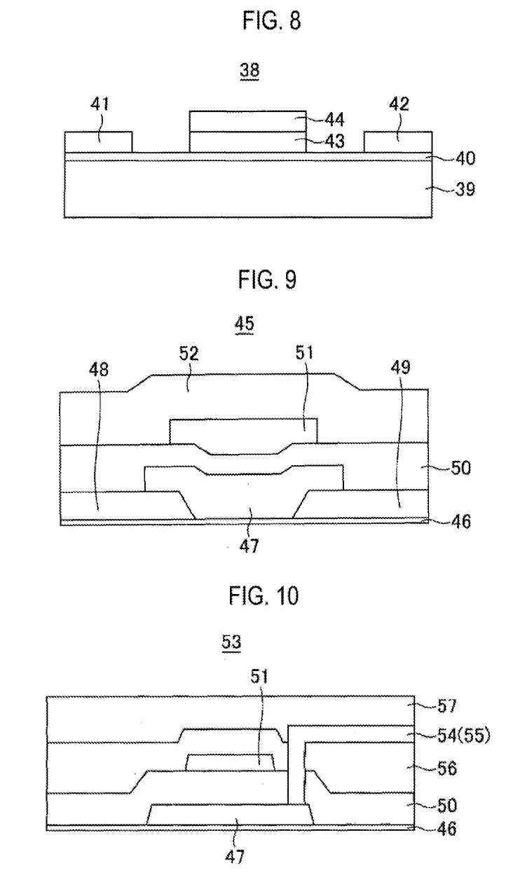

FIG. 8 is a sectional view schematically illustrating the configuration of an example of a top-gated thin-film transistor structure where an organic semiconductor film according to each embodiment of the present disclosure is applied.

Referring to FIG. 8, thin-film transistor structure 38 comprises channel layer 40 formed on substrate 39; source electrode 41 and drain electrode 42 formed on channel layer 40 so as to be separated from each other; gate-insulating film 43 formed, on channel layer 40, between source electrode 41 and drain electrode 42; and gate electrode 44 formed on gate-insulating film 43. Channel layer 40 is comprised of an organic semiconductor film according to the first or second embodiment. In addition, a passivation layer may be interposed between substrate 39 and channel layer 40.

FIG. 9 is a sectional view schematically illustrating the configuration of the first variant example of a top-gated thin-film transistor structure where an organic semiconductor film according to each embodiment of the present disclosure is applied.

Referring to FIG. 9, thin-film transistor structure 45 comprises undercoat layer 46 formed on a substrate (not shown); channel layer 47 formed on a part of undercoat layer 46; source electrode 48 and drain electrode 49 respectively formed on undercoat layer 46 on opposite sides of channel layer 47; gate-insulating film 50 formed so as to cover channel layer 47, source electrode 48 and drain electrode 49; gate electrode 51 formed on gate-insulating film 50 to be located directly above channel layer 47; and passivation layer 52 formed so as to cover gate electrode 51 and gate-insulating film 50. Channel layer 47 is comprised of an organic semiconductor film according to the first or second embodiment.

FIG. 10 is a sectional view schematically illustrating the configuration of the second variant example of a top-gated thin-film transistor structure where an organic semiconductor film according to each embodiment of the present disclosure is applied.

Referring to FIG. 10, thin-film transistor structure 53 comprises undercoat layer 46 formed on a substrate (not shown); channel layer 47 formed on a part of undercoat layer 46; source electrode 54 and drain electrode 55 connected to channel layer 47; gate-insulating film 50 formed so as to cover undercoat layer 46 and channel layer 47; gate electrode 51 formed on gate-insulating film 50 to be located directly above channel layer 47; interlayer insulating film 56 formed so as to cover gate electrode 51 and gate-insulating film 50; and passivation layer 57 formed so as to cover interlayer insulating film 56, source electrode 54 and drain electrode 55. Channel layer 47 is comprised of an organic semiconductor film according to the first or second embodiment.

As the organic semiconductor film of the above embodiments is comprised of a monolayer of pseudo graphene structure, channel layers 17, 24, 31, 40 and 47 can be formed as a thin-film several Å thick. Since this organic semiconductor film has a band gap, thin-film transistor structures 14, 20, 27, 35, 38, 45 and 53 can perform the switching operation.

In thin-film transistor structures 14, 20, 27, 35, 38, 45, and 53, substrates 21 and 39 are preferably comprised of a flexible member, for example, a glass foil, a metal foil (e.g., stainless steel), or a resin substrate (e.g., a polycarbonate, a polyethylene terephthalate, a polyethylene naphthalate, a cyclic-olefin polymer (ATRON, APEL, ZEONEX), a polyarylate, an aromatic polyether ketone, an aromatic polyether sulfone, a fully aromatic polyketone or a polyimide).

In addition, gate-insulating films 16, 23, 30, 43, and 50 are preferably comprised of a polymeric material (e.g., a polychloroprene, a polyethylene terephthalate, a polyoxymethylene, polyvinyl chloride, a polyvinylidene fluoride, a cyanoethyl-pullulan, a polymethylmethacrylate, a polysulfone, a polycarbonate, a polyvinyl phenol, a polystyrene or a polyimide) or an inorganic material (e.g., SiO₂, SiN, Al₂O₃, HfO₂ or BN).

In addition, gate electrodes 15, 22, 29, 44, and 51, source electrodes 18, 25, 32, 41, and 48 and drain electrodes 19, 26, 33, 42, and 49 are preferably comprised of a metal (e.g., Ag, Al, Cu, Pt, Au, Ni, Co, Pd, Ti or Cr), an oxide conductor (e.g., ITO (indium tin oxide) or ZnO (zinc oxide)), or an organic material conductor (e.g., a conductive polymer).

While the present disclosure has been described above in connection with particular embodiments, the present disclosure is not limited to those embodiments.

For example, although organic semiconductor films according to the above embodiments were manufactured using ultrahigh vacuum film forming device 10 of FIG. 1, these organic semiconductor films may also be manufactured using a conventional vapor deposition film forming device (for example, a low vacuum film forming device or a resistance heating film forming device).

The present application claims priority to Japanese Patent Application No. 2013-116758 filed on Jun. 3, 2013, the entire contents of which are incorporated herein by reference.

### EXPLANATION OF REFERENCE NUMERALS

14: thin-film transistor structure

17: channel layer

1. An organic semiconductor film having a pseudo graphene structure formed by contiguous extension of a two-dimensional network structure represented by formula (I).

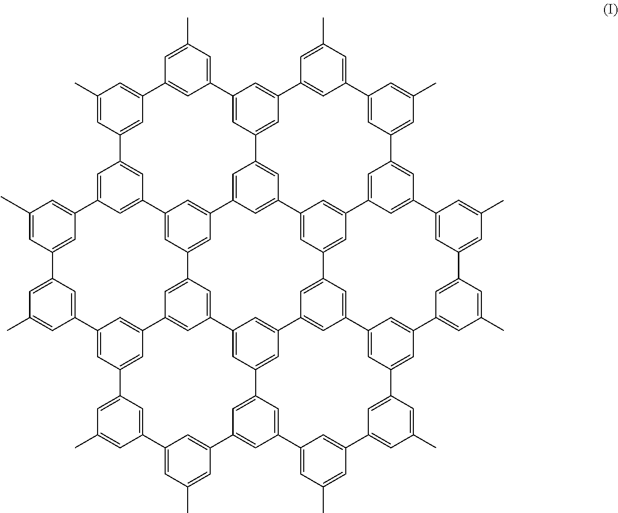

2. The organic semiconductor film of claim 1, wherein the pseudo graphene structure is comprised of a monolayer of the two-dimensional network structure.

3. An organic semiconductor film having a pseudo graphene structure formed by contiguous extension of a two-dimensional network structure represented by formula (II).

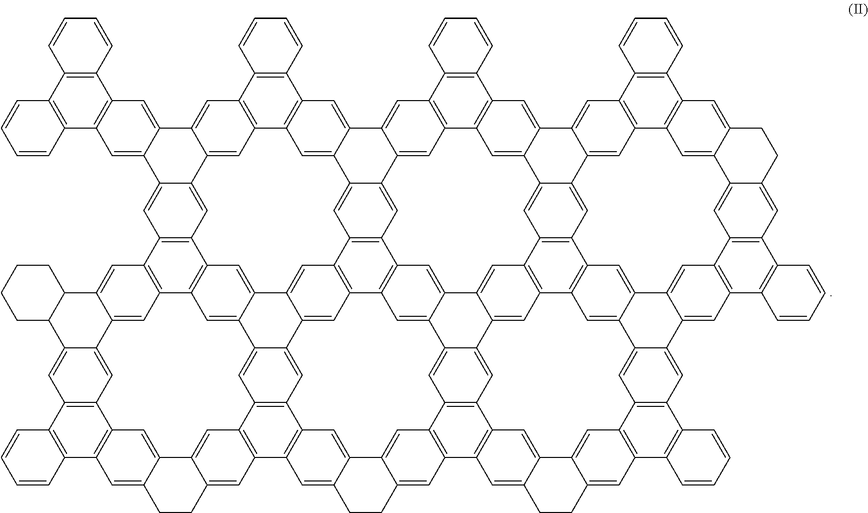

4. The organic semiconductor film of claim 3, wherein the pseudo graphene structure is comprised of a monolayer of the two-dimensional network structure.

5. A method for preparing an organic semiconductor film having two-dimensional network structure in the organic semiconductor film of claim 1, the two-dimensional network structure being formed by polymerizing a plurality of 5,5′,5″,5′″,5″″,5′″″-hexabromocyclohexa-m-phenylene (CHP), the CHP having bromine at its side chains.

6. The method for preparing an organic semiconductor film of claim 5, wherein the two-dimensional network structure is formed by depositing the plurality of CHPs on the surface of a single-crystalline metal having catalytic activity.

7. The method for preparing an organic semiconductor film of claim 6, wherein, when the single-crystalline metal has a face centered cubic lattice, the surface of the single-crystalline metal is composed of (111) plane of the face centered cubic lattice.

8. The method for preparing an organic semiconductor film of claim 6, wherein, when the single-crystalline metal has a hexagonal close packed structure, the surface of the single-crystalline metal is composed of (0001) plane of the hexagonal close packed structure.

9. The method for preparing an organic semiconductor film of claim 5, wherein the two-dimensional network structure is formed by depositing the plurality of CHPs on the surface of a polycrystalline metal comprised of grains having catalytic activity.

10. The method for preparing an organic semiconductor film of claim 9, wherein, when the polycrystalline metal has a face centered cubic lattice, the surface of the grains is composed of (111) plane of the face centered cubic lattice.

11. The method for preparing an organic semiconductor film of claim 9, wherein, when the polycrystalline metal has a hexagonal close packed structure, the surface of the grains is composed of (0001) plane of the hexagonal close packed structure.

12. A method for preparing an organic semiconductor film having the two-dimensional network structure in the organic semiconductor film of claim 3, the two-dimensional network structure being formed by polymerizing a plurality of 2,3,6,7,10,11-hexabromotriphenylene (HBTP).

13. The method for preparing an organic semiconductor film of claim 12, wherein the two-dimensional network structure is formed by depositing the plurality of HBTPs on the surface of a single-crystalline metal having catalytic activity.

14. The method for preparing an organic semiconductor film of claim 13, wherein, when the single-crystalline metal has a face centered cubic lattice, the surface of the single-crystalline metal is composed of (111) plane of the face centered cubic lattice.

15. The method for preparing an organic semiconductor film of claim 13, wherein, when the single-crystalline metal has a hexagonal close packed structure, the surface of the single-crystalline metal is composed of (0001) plane of the hexagonal close packed structure.

16. The method for preparing an organic semiconductor film of claim 12, wherein the two-dimensional network structure is formed by depositing the plurality of HBTPs on the surface of a polycrystalline metal comprised of grains having catalytic activity.

17. The method for preparing an organic semiconductor film of claim 16, wherein, when the polycrystalline metal has a face centered cubic lattice, the surface of the grains is composed of (111) plane of the face centered cubic lattice.

18. The method for preparing an organic semiconductor film of claim 16, wherein, when the polycrystalline metal has a hexagonal close packed structure, the surface of the grains is composed of (0001) plane of the hexagonal close packed structure.

19. A transistor structure characterized in that the organic semiconductor film of claim 1 is used for channels.
